(19) United States
(12) Patent Application Publication
(10) Pub. No.: US 2020/0376468 A1
(43) Pub. Date: Dec. 3, 2020

(54) METHOD FOR PREPARING A CATALYST AND METHOD FOR PRODUCING 1,4-BUTANEDIOL AND/OR TETRAHYDROFURAN FROM FURAN

(71) Applicant: SHELL OIL COMPANY, HOUSTON, TX (US)

(72) Inventors: Sipke Hidde WADMAN, Amsterdam (NL); Jeroen Karel VAN GELDER, Amersfoort (NL); Jean-Paul Andre Marie Joseph Ghislain LANGE, Amsterdam (NL)

(21) Appl. No.: 16/636,778

(22) PCT Filed: Aug. 8, 2018
(86) PCT No.: PCT/EP2018/071523

§ 371 Date: Feb. 5, 2020

Related U.S. Application Data
(60) Provisional application No. 62/543,472, filed on Aug. 10, 2017.

(51) Int. Cl. B01J 23/656; B01J 23/44; B01J 21/18; B01J 35/02; B01J 37/02; C07C 29/17; C07D 307/08

(52) U.S. Cl. CPC B01J 23/6567; B01J 23/44; B01J 21/18; B01J 35/026; C07D 307/08; B01J 37/024; B01J 37/0205; C07C 29/172; B01J 37/0236

(57) ABSTRACT

A method for preparing a metal-impregnated, carbon-supported catalyst composition is provided. The method comprises providing a carbon support particle having a smallest dimension of greater than 0.5 millimeters; contacting the carbon support particle with a basic aqueous impregnation solution comprising abase having a pK_{b} of at most 9 and at least one first metal-containing compound, wherein the first metal-containing compound comprises at least one first metal selected from groups 8, 9 and 10 of the periodic table, to form a first metal-impregnated carbon support particle; and drying the first metal-impregnated carbon support particle.

### BACKGROUND

Furan and its derivatives are useful precursors for industrial chemicals in the areas of, for example, pharmaceuticals, herbicides and polymers. Furan may be converted into tetrahydrofuran (THF) and 1,4-butanediol (1,4-BDO). THF and 1,4-BDO are valuable chemicals used industrially as solvents and in the production of elastic fibres such as elastane/spandex, polybutyrate terephthalate and derivatives of gamma butyrolactone.

There are numerous methods disclosed in the art for making THF and 1,4-BDO. For example, U.S. Pat. No. 5,905,159 discloses a process in which furan is converted as a reaction mixture with water and in the presence of hydrogen, but in the absence of a water-soluble acid, in a single stage over a hydrogenation catalyst to THF and 1,4-BDO. The hydrogenation catalyst of U.S. Pat. No. 5,905,159 contains at least one element of groups 1, 5, 6, 7 or 8 of the periodic table, with the restriction that the catalyst does not contain nickel alone. The catalysts taught in U.S. Pat. No. 5,905,159 generally contain two metals with most containing rhenium as a promoter. The most preferred catalyst taught in U.S. Pat. No. 5,905,159 for the process is rhenium/ruthenium on active carbon.

WO2016087508 describes a process for the preparation of 1,4-BDO and THF in which furan is contacted with hydrogen and water in the presence of a supported catalyst comprising rhenium and palladium in a weight ratio of at least 1:1 and a total combined weight of rhenium and palladium in the range of from 0.01 to 20 wt %. WO2016087508 further describes that such a catalyst is highly effective in the conversion of furan to 1,4-BDO and THF without the production of large amounts of n-butanol as a side product.

Conventionally, the catalyst used in the preparation of 1,4-BDO and THF from furan is a metal-impregnated, carbon-supported catalyst in the form of a fine particulate. The usual method of preparation is to add to the support an aqueous solution of the active metal component in the form of a soluble decomposable salt. After impregnation is complete, the excess solution, if any, is decanted and the impregnated support is dried to remove water and thereafter optionally calcined. Due to the fine particulate nature of the support, any non-uniform distribution of metal on the carbon support particles resulting from this preparation method has been inconsequential. However, when larger carbon support particles are used, this method of impregnation results in an unequal “shell-type” distribution of the impregnated metal on the carbon support, which is problematic.

### SUMMARY

A method for preparing a metal-impregnated, carbon-supported catalyst composition is provided. The method comprises providing a carbon support particle having a smallest dimension of greater than 0.5 millimeters; contacting the carbon support particle with a basic aqueous impregnation solution comprising a base having a pK_{b} of at most 9 and at least one first metal-containing compound, wherein the first metal-containing compound comprises at least one first metal selected from groups 8, 9 and 10 of the periodic table, to form a first metal-impregnated carbon support particle; and drying the first metal-impregnated carbon support particle.

Also provided is a method for the preparation of 1,4-butanediol and/or tetrahydrofuran that comprises contacting furan, hydrogen and optionally water in the presence of a metal-impregnated, carbon-supported catalyst composition prepared in accordance with the above-mentioned method.

The features and advantages of the present disclosure will be apparent to those skilled in the art. While numerous changes may be made by those skilled in the art, such changes are within the spirit of the invention.

### DETAILED DESCRIPTION

It has been discovered that metal maldistribution on larger carbon support particles (i.e., carbon support particles having a smallest dimension of greater than 0.5 millimeters (“mm”)) may be minimized or avoided by impregnating the carbon support particles with a basic aqueous impregnation solution comprising a base having a pK_{b} of at most 9 and at least one first metal-containing compound, wherein the first metal-containing compound comprises at least one first metal selected from groups 8, 9 and 10 of the periodic table. The metal-impregnated carbon support particle is then dried and optionally calcined.

Without wishing to be bound by any particular theory or mechanism, it is believed that by using a basic aqueous impregnation solution comprising a base having a pK_{b} of at most 9, at least a portion of the acidic sites present on the outer surface of the carbon support particle are neutralized, which advantageously then allows for improved, homogenous distribution of the catalytic metal.

Carbon support particles suitable for use herein are not particularly limited and may include any such material having a smallest dimension of greater than 0.5 mm. Preferably, the carbon support particle comprises activated carbon, such as extruded activated carbon, which can be sourced from commercial suppliers known to the skilled person. Other examples of suitable carbon support particles include carbon black, graphite, graphene based or structure carbons, such as carbon nanotubes and carbon nanofibers, provided that such materials are bound or cross-linked in a suitable manner to form particles having a smallest dimension of greater than 0.5 mm.

Suitable carbon support particles may include particles having any of various regular or irregular shapes, such as cylinders, spheres, tablets, discs, rings, stars, or other shapes, provided that the smallest dimension is greater than 0.5 mm. For example, a carbon support particle may have dimensions such as diameter, length or width of 0.5 mm to 10 mm, e.g., from 1 mm to 9 mm, or from 2 mm to 8 mm. Preferably, the particles' largest dimension is from 2 mm to 9 mm, e.g., from 3 mm to about 8 mm or from 4 mm to 7 mm. Surface areas available for suitable carbon support particles, as measured by the BET (Brunauer, Emmett, and Teller) method, may generally be between 100 m²/g and 5000 m²/g, e.g., from 200 m²/g to 2000 m²/g or from 400 m²/g to 1000 m²/g. Also, the pore volume of the support material may generally range from 0.4 mL/g to 1.4 mL/g, e.g., from 0.6 mL/g to 1.2 mL/g or from 0.8 mL/g to 1.0 mL/g.

A basic aqueous impregnation solution, used to make a metal-impregnated carbon support particle, comprises a base having a pK_{b} of at most 9 and at least one first metal-containing compound, wherein the first metal-containing compound comprises at least one first metal selected from groups 8, 9 and 10 of the periodic table. With respect to suitable bases, such bases have a pK_{b} of at most 9, when measured in water at 25° C., or a pK_{b} of less than 9, or a pK_{b} of at most 7, or a pK_{b} of at most 5. Examples of suitable bases include ammonia, as well as oxides, hydroxides, phosphates (PO₄³⁻) and alcoholates of alkali and/or alkali earth metals. Typically, the base is present in the basic aqueous impregnation solution in an amount from 1 to 30 wt. %, or from 5 to 15 wt. %, or from 8 to 12 wt. %, or from 0.5 to 15 mM, or from 2 to 10 mM, or from 4 to 8 mM. Suitably, the pH of the basic aqueous impregnation solution is typically from 8 to 10, or from 9 to 12 or from 10 to 11. Optionally, a basic aqueous impregnation solution may further comprise an acid, such as citric acid, and have a pH from 5 to 10.

A basic aqueous impregnation solution further comprises at least one first metal-containing compound, wherein the first metal-containing compound comprises at least one first metal selected from groups 8, 9 and 10 of the periodic table. The at least one first metal selected from groups 8, 9 and 10 of the periodic table may be suitably selected from a group consisting of ruthenium, rhodium, palladium, platinum and iridium. Further, the basic aqueous impregnation solution may comprise a single such metal, or a combination of such metals. Examples of such combinations include, but are not limited to, for example, ruthenium and palladium or ruthenium and platinum.

In preparing a basic aqueous impregnation solution, at least one first metal-containing compound comprising at least one of the abovementioned metal(s) is selected. Examples of suitable first metal-containing compounds include, but are not limited to, a salt or a complex of at least one first metal selected from groups 8, 9 and 10 of the periodic table. The salt or complex may comprise anions such as, but not limited to, nitrate, chloride, acetylacetonate, acetate, etc., optionally in combination with neutral ligands, such as NO and NH₃. The first metal-containing compound needs to be soluble in the aqueous solvent, such that a sufficient amount of the at least one first metal from groups 8, 9 and 10 of the periodic table is present in a dissolved form in the basic aqueous impregnation solution for impregnating the carbon support particle. The meaning of ‘sufficient amount’ is discussed below.

With regards to suitable aqueous solvents, any aqueous solvent in which all of the components of the impregnation solution are miscible may be used. In addition, suitable aqueous solvents should also be capable of being removed in subsequent steps, either by a washing, volatilizing or oxidation procedure, or the like. For example, the aqueous solvent may be water, or a combination of water and a water-soluble co-solvent, such as an alcohol (e.g., methanol or ethanol), glycol (e.g., ethylene glycol or propylene glycol), or a ketone (e.g., acetone). Typically, the amount of aqueous solvent present in the basic aqueous impregnation solution may vary within wide ranges, and is typically at least 30 wt. %, or at least 50 wt. %, or at least 70 wt. %, or at least 90 wt. %.

To prepare the basic aqueous impregnation solution, the total amount of the abovementioned metal in the impregnation solution needs to be known; such amount being referred to herein as a/the ‘sufficient amount’. The sufficient amount is dependent on the amount of carbon support particles to be impregnated, such that, after contacting the carbon support particles with the basic aqueous impregnation solution, the total weight percentage of the at least one first metal from groups 8, 9 and 10 of the periodic table impregnated on the carbon support particle, compared to the total weight of the resultant catalyst composition, is preferably at least 0.01 wt. % metal, or at least 0.03 wt. % metal, or at least 0.1 wt. % metal, or at least 0.3 wt. % metal, or at least 1 wt. % metal or at least 3 wt. % metal and preferably at most 10 wt. % metal, or at most 7 wt. % metal or at most 5 wt. % metal.

With the knowledge of the ‘sufficient amount’, a volume of the basic aqueous impregnation solution is prepared. The volume of basic aqueous impregnation solution may be such that carbon support particles are impregnated until a point of incipient wetness of the support particles has been reached. Alternatively, a larger volume may be used and the surplus of solution may be removed from the wet carbon support particles, for example by decantation. Further, the volume of impregnation solution may be such that it corresponds to 90-110%, preferably 95-100%, of the pore volume of the carbon support particles. The ‘sufficient amount’ of the basic aqueous impregnation solution is contacted with a predetermined amount of the carbon support particles, and typically, a brief mixing step is then performed to enhance the even contact of the basic aqueous impregnation solution with the carbon support particles. Suitably, during and immediately after the brief mixing step, the basic aqueous impregnation solution becomes evenly distributed over the carbon support particle surface area, and as the aqueous solvent is removed by drying, the dissolved metal in the basic aqueous impregnation solution begins to impregnate on the carbon support particle. The principles underlying such absorption/deposition/impregnation process, otherwise known as incipient wetness impregnation, is known to the skilled person. Suitably, other methods of metal absorption/deposition/impregnation that are known to the skilled person may be also used. At the end of these steps, a metal-impregnated carbon support particle is formed.

After impregnation, the metal-impregnated carbon support particle may be dried, typically at a temperature of no greater than 400° C., so that the processes of calcining and metal sintering, known to the skilled person, are avoided. Preferably, the drying temperature is at most 300° C., or at most 225° C., or at most 150° C., and or at most 100° C., and suitably at a temperature of at least 20° C., or at least 50° C., or at least 70° C. Suitably, if the drying temperature is at most 300° C., typically the drying time may be no longer than 30 minutes. Suitably, if the drying temperature is at most 225° C., typically the drying time may be no longer than 2 hours. Suitably, if the drying temperature is at most 150° C. or less, typically the drying time may be overnight. Suitably, the atmospheric composition during drying is the same as ambient atmospheric composition. However, drying under reduced atmosphere and temperature lower than ambient temperature is also possible and known to persons skilled in the art.

As would be recognized by one skilled in the art, if drying is conducted at a lower temperature, a longer period of time is generally required and likewise, if drying is conducted at a higher temperature, less time is typically required. Although it is provided herein that drying should generally be conducted at a temperature in a range of from 20° C. to no greater than 400° C., for a period of time from a few minutes to 12 hours, and at atmospheric pressure, the present disclosure is nevertheless independent of the manner by which such drying is conducted. Thus, variations in drying known in the art, such as holding at one temperature for a certain period of time and then raising the temperature to a second temperature over the course of a second period of time, are contemplated by the present disclosure. Furthermore, the equipment used for such drying may use a static or flowing atmosphere of such gases to effect reduction, preferably a flowing atmosphere.

Optionally, in addition to the at least one first metal selected from groups 8, 9 and 10 of the periodic table, the metal-impregnated, carbon-supported catalyst composition may further comprise at least one second metal selected from groups 6 and 7 of the periodic table. The at least one second metal may be suitably selected from a group consisting of rhenium, molybdenum and tungsten.

The at least one second metal, if present, may be deposited either prior to, coincidentally with, or subsequent to the deposition of the at least one first metal. For example, an aqueous impregnation solution comprising at least one second metal-containing compound may be prepared and brought into contact with a carbon support particle prior to contacting the support with the basic aqueous impregnation solution. Alternatively, an aqueous impregnation solution comprising at least one second metal-containing compound may be brought into contact with a carbon support particle subsequent to contacting the support with the basic aqueous impregnation solution and drying. Suitably, to deposit a first and second metal coincidentally, a first metal-containing compound and a second metal-containing compound may both be included in a basic aqueous impregnation solution, provided that the base having a pK_{b} of at most 9 is not ammonia and that the pH of the basic aqueous impregnation solution is at least 8.

Examples of suitable second metal-containing compounds include, but are not limited to, a salt or a complex of at least one second metal selected from groups 6 and 7 of the periodic table. The salt or complex may consist of oxy, hydro and oxyhydroxy species of the group 6 or 7 metal, optionally as anion of an alkali or alkali earth salt. Further, the aqueous impregnation solution may comprise a single such metal, or a combination of such metals. The second metal-containing compound needs to be soluble in the aqueous solvent, such that a sufficient amount of the at least one second metal from groups 6 and 7 of the periodic table is present in a dissolved form in the impregnation solution for impregnating the carbon support particle. The sufficient amount is dependent on the amount of carbon support particles to be impregnated, such that, after contacting the carbon support particles with the aqueous impregnation solution, the total weight percentage of the at least one second metal from groups 6 and 7 of the periodic table impregnated on the carbon support particle, compared to the total weight of the resultant catalyst composition, is preferably at least 0.2 wt. % metal, or at least 0.5 wt. % metal, or at least 1 wt. % metal, or at least 2 wt. % metal, and preferably at most 10 wt. % metal, or at most 7 wt. % metal or at most 5 wt. % metal.

In one embodiment, the first metal-containing compound comprises palladium and the second metal-containing compound comprises rhenium. Suitably, the rhenium and palladium are present on the finished metal-impregnated, carbon-supported catalyst composition in a weight ratio of at least 1:1. This ratio is the weight ratio of the metals considered as elements in the catalyst with which the furan is brought into contact. More preferably, the weight ratio of rhenium:palladium is at least 5:1, more preferably at least 10:1, even more preferably at least 20:1. Further advantages, such as increased yields of BDO may be obtained by even higher weight ratios, for example at least 50:1.

Typically, the total amount of metal (considered as the element) on the finished metal-impregnated, carbon-supported catalyst composition may vary within wide ranges, and may be of from 0.01 to 20 wt %, from 0.1 to 10 wt % or from 0.5 to 5 wt % on the basis of the total weight of the catalyst. Suitably, the total amount of metal is typically at least 0.01 wt %, or at least 0.03 wt %, or at least 0.1 wt %, or at least 0.3 wt %, or at least 1.0 wt %, or at least 3.0 wt %. Further, the total amount of metal is typically at most 20 wt %, or at most 15 wt %, or at most 10 wt %.

Optionally, a base may be deposited on the carbon support particle prior to depositing a first metal on the carbon support particle. For example, a solution comprising a base having a pK_{b} of at most 9, when measured in water at 25° C., or a pK_{b} of less than 9, or a pK_{b} of at most 7, or a pK_{b} of at most 5, may be prepared and brought into contact with a carbon support particle prior to contacting the support with a basic aqueous impregnation solution.

Also provided is a method for the preparation of 1,4-butanediol and/or tetrahydrofuran that comprises contacting furan, hydrogen and optionally water in the presence of a metal-impregnated, carbon-supported catalyst composition, prepared in accordance with the above-mentioned methods. The furan may be contacted with hydrogen either in the gas or the liquid phase.

Suitable conditions for the production of 1,4-BDO and THF from furan include gas- or liquid phase conditions in the absence or presence of gas or liquid diluent. For liquid phase condition, an inert non-polar or moderately polar solvent, such as a hydrocarbon or oxygenate, can be used. However, such a process will mainly form THF. In order for 1,4-BDO to be produced, water must be present in the reaction mixture. Further conditions include a temperature in the range of from 25 to 250° C., a pressure of from 0.1 to 15 MPa and a H₂:furan molar ratio in the range of from 0.2:1 to 100:1, preferably in the range of from 0.2:1 to 10:1 and most preferably in the range from 1:1 to 3:1.

Alternative suitable conditions for the production of a mixture of BDO and THF include co-feeding water as a gas or liquid at a water:furan molar ratio in the range of from 0.2:1 to 100:1, preferably in the range of 1:1 to 20:1 and most preferably 3:1 to 10:1. In this embodiment, further suitable conditions include the use of a solvent comprising water and/or oxygenates, preferably the reaction product (THF and/or BDO) or eventually by-products (1-butanol), a temperature in the range of from 100 to 350° C., preferably 120 to 250° C., most preferably 150-200° C., a pressure of from 0.1 to 15 MPa, preferably 1-10 MPa and most preferably 3-7 MPa and a H₂:furan molar ratio in the range of from 0.2:1 to 100:1, preferably in the range of from 1:1 to 10:1, most preferably 2:1 to 5:1.

Having generally described the invention, a further understanding may be obtained by reference to the following examples, which are provided for purposes of illustration only and are not intended to be limiting unless otherwise specified.

### EXAMPLES

Preparation of Catalysts

For Examples 1-4 and Comparative Examples 1-2, carbon support particles (RX4-extra from Cabot) having a BET surface area of about 1200 m²/g, a pore volume of 0.61 ml/g (mainly consisting of micropores), and a bulk density of 0.34 ml/g were used. The carbon support particles were cylinders having a diameter of 4 mm. All impregnations were carried out at incipient wetness, using a solution volume that equals the pore volume of the carbon support particles to be impregnated.

### Examples 1A-1D: Preparation of Pd Catalyst

A basic aqueous impregnation solution comprising ammonia and a palladium-containing compound (a first metal-containing compound) was prepared by dissolving the target amount of either tetraamine palladium nitrate (Pd(NH₃)₄(NO₃)₂) (Examples 1A, 1B and 1D) or palladium (II) nitrate (Pd(NO₃)₂) (Example 1C) into the target amount of aqueous ammonia solution (˜12 w % NH₃) and homogenizing the solution for 30 seconds. For Examples 1C and 1D, the basic aqueous impregnation solution further comprised citric acid, which was added to the aqueous Pd solution and the amount of ammonia was adjusted such that the solution had a pH of 5. The carbon support particles were loaded into a glass jar and the basic aqueous impregnation solution was then poured on the carbon support particles and homogenized using a rotary mixer for one hour. The palladium impregnated carbon support particles were then transferred to a rotary bowl equipped with baffles and dried at 60° C. by means of an air dryer that heats the external wall of the bowl. The dried, palladium impregnated carbon support particles were finally transferred to a porcelain dish and dried in an oven set at 120° C. for 2 hours in static air.

### Examples 2A-2C: Preparation of Re/Pd Catalyst

In Examples 2A-2C, dried, palladium impregnated carbon support particles, which were prepared in accordance with Example 1, were used for subsequent impregnation with rhenium.

An aqueous impregnation solution comprising a rhenium-containing compound was prepared by dissolving the target amount of perrhenic acid (HReO₄) (a second metal-containing compound) into the target amount of demineralized water and homogenizing the solution for 30 seconds. Dried, palladium impregnated carbon support particles (prepared according to Example 1) were loaded into a glass jar and the aqueous impregnation solution was then poured on the carbon support particles and homogenized using a rotary mixer for one hour. The palladium and rhenium impregnated carbon support particles were then transferred to a rotary bowl equipped with baffles and dried at 60° C. by means of an air dryer that heats the external wall of the bowl. The dried, palladium and rhenium impregnated carbon support particles were then transferred to a porcelain dish and dried in an oven set at 120° C. for 2 hours in static air.

### Example 3: Preparation of Pd/Re Catalyst

In Example 3, the impregnation sequence of Example 2 was inversed. That is to say, carbon support particles were first impregnated with an aqueous impregnation solution comprising a rhenium-containing compound and dried, as described in Example 2, then the dried, rhenium impregnated carbon support particles were impregnated with a basic aqueous impregnation solution comprising ammonia and a palladium-containing compound and dried, as described in Example 1.

### Example 4: Preparation of Pd/NH₃/Re Catalyst

In Example 4, the impregnation sequence of Example 3 was modified to include a base neutralisation step prior to impregnating the dried, rhenium impregnated carbon support particles with a basic aqueous impregnation solution comprising ammonia and a palladium-containing compound.

For base neutralization, a target amount of aqueous ammonia solution (˜12 w % NH₃) was prepared. Dried, rhenium impregnated carbon support particles (prepared as described in Example 2) were loaded into a glass jar and the solution comprising the base was then poured on the rhenium impregnated carbon support particles and homogenized using a rotary mixer for one hour. The impregnated carbon support particles were then transferred to a rotary bowl equipped with baffles and dried at 60° C. by means of an air dryer that heats the external wall of the bowl. The dried, rhenium impregnated carbon support particles were then transferred to a porcelain dish and dried in an oven set at 120° C. for 2 hours in static air.

Subsequently, the dried, rhenium impregnated carbon support particles were impregnated with a basic aqueous impregnation solution comprising ammonia and a palladium-containing compound and dried, as described in Example 1.

### Comparative Examples 1E-1G: Preparation of Pd Catalyst

In Comparative Examples 1E-1G, the catalyst preparation of Example 1 was modified so that the aqueous impregnation solution comprising a palladium-containing compound did not contain any NH₃, but did contain as acid additive, oxalic acid, HCl, or acetic acid. In addition, the palladium-containing compound used was dihydrogen palladium tetrachloride (H₂PdCl₄), rather than tetraamine palladium nitrate (Pd(NH₃)₄(NO₃)₂), as H₂PdCl₄ is more compatible with acidic medium.

### Comparative Examples 2D-2E: Preparation of Pd+Re Catalyst

In Comparative Examples 2D-2E, the catalyst preparation of Example 1 was modified so that the aqueous impregnation solution comprising a palladium-containing compound did not contain any NH₃, but did further comprise a rhenium-containing compound, perrhenic acid (HReO₄). In addition, the palladium-containing compound used was dihydrogen palladium tetrachloride (H₂PdCl₄), rather than tetraamine palladium nitrate (Pd(NH₃)₄(NO₃)₂₁, as H₂PdCl₄ is more compatible with acidic medium.

XPS Analysis and Results

XPS Measurements were performed using the Kratos Axis Nova instrument using 15 kV Al Kα source with sample neutralization. All samples were in vacuum for about 15 hours before the first measurement. For each sample, two catalyst particles were selected and broken. One part was put on the side to measure the external surface of the particle and another part was placed with the freshly created surface facing up to measure on the internal surface of the particle.

The samples were analysed and the resulting Pd/C atomic ratio obtained for external and internal measurements was compared to the ideal ratio expected from homogeneous Pd distribution throughout the sample. Considering the molecular weight of Pd (106.4 g/mol) and C (12.0 g/mol), the ideal Pd/C can be calculated as w % Pd*12.0/106.4.

TABLE 1
XPS analysis of metal distribution of Pd for Examples
1A-1D and Comparative Examples 1E-1G.

| | | | | External/ | Internal/ |
|---|---|---|---|---|---|
| | Pd | | | Ideal | Ideal |
| Example | w % | Pd source | additive | Pd/C | Pd/C |
| 1A | 1.0 | Pd(NH₃)₄(NO₃)₂ | NH₃ | 4-4.5 | 0.2-1 |
| 1B | 0.2 | Pd(NH₃)₄(NO₃)₂ | NH₃ | 3-6 | 1.0-1.5 |
| 1C | 0.2 | Pd(NO₃)₂ | citric | 3-6 | na |
| | | | acid + NH₃ | | |
| | | | (pH = 5) | | |
| 1D | 0.2 | Pd(NH₃)₄(NO₃)₂ | citric | 3-6 | na |
| | | | acid + NH₃ | | |
| | | | (pH = 5) | | |
| Comp. 1E | 0.2 | H₂PdCl₄ | oxalic acid | 44-120 | na |
| Comp. 1F | 0.2 | H₂PdCl₄ | HCl | 14-120 | na |
| Comp. 1G | 0.2 | H₂PdCl₄ | acetic acid | 170-315 | na |

TABLE 2
XPS analysis of metal distribution of Pd for Examples
2A-2C and Comparative Examples 2D-2E.

| | Pd | | External/ | Internal/ |
|---|---|---|---|---|
| Example | w % | Re w % | Ideal Pd/C | Ideal Pd/C |
| 2A | 1.0 | 4.0 | 4.5 | 0.6 |
| 2B | 0.2 | 4.0 | 3.5 | 0.4 |
| 3 | 0.2 | 4.0 | 3 | 0.1 |
| 4 | 0.2 | 4.0 | 3.5 | 0.2 |
| 2C | 0.2 | 6.0 | 3.5 | 0.3 |
| Comp. 2D | 0.2 | 4.0 | 140-220 | na |
| Comp. 2E | 0.2 | 4.0 | 340-600 | na |

The results reported in Tables 1-2 show that the samples prepared in accordance with the present disclosure (Examples 1-4) have a much better Pd distribution throughout the particle since the external Pd/C ratio is only 3-6× the ideal ratio while it is 40× up to 600× the ideal ratio for Comparative Examples 1-2.

When available, XPS analysis of the internal surface of the particle prepared in accordance with the present disclosure revealed Pd/C ratios that are close to the ratio expected for ideal distribution, typically between 0.2× and 0.6× the ideal ratio.

Catalytic Evaluation

For Example 5 and Comparative Example 3, carbon support particles (RX4-extra from Cabot) having a BET surface area of about 1200 m²/g, a pore volume of 0.61 ml/g (mainly consisting of micropores), and a bulk density of 0.34 ml/g were used. The carbon support particles were cylinders having a diameter of 4 mm. All impregnations were carried out at incipient wetness, using a solution volume that equals the pore volume of the carbon support particles to be impregnated.

### Example 5: Preparation of Pd/Re Catalyst for Catalytic Evaluation

For Example 5, a basic aqueous impregnation solution comprising ammonia and a palladium-containing compound was prepared by dissolving the target amount of tetraamine palladium nitrate (Pd(NH₃)₄(NO₃)₂) into the target amount of aqueous ammonia solution (˜12 w % NH₃) and homogenizing the solution for 30 seconds. The carbon support particles were loaded into a glass jar and the basic aqueous impregnation solution was then poured on the carbon support particles and homogenized using a rotary mixer for one hour. The palladium impregnated carbon support particles were then transferred to a rotary bowl equipped with baffles and dried at 60° C. by means of an air dryer that heats the external wall of the bowl. The dried, palladium impregnated carbon support particles were transferred to a porcelain dish and dried in an oven set at 120° C. for 2 hours in static air.

Subsequently, the dried, palladium impregnated carbon support particles were contacted with an aqueous impregnation solution comprising a rhenium-containing compound, which was prepared by dissolving the target amount of perrhenic acid (HReO₄) into the target amount of demineralized water and homogenizing the solution for 30 seconds. The dried, palladium impregnated carbon support particles were loaded into a glass jar and the aqueous impregnation solution was poured on the carbon support particles and homogenized using a rotary mixer for one hour. The palladium and rhenium impregnated carbon support particles were then transferred to a rotary bowl equipped with baffles and dried at 60° C. by means of an air dryer that heats the external wall of the bowl. The dried, palladium and rhenium impregnated carbon support particles were then transferred to a porcelain dish and dried in an oven set at 120° C. for 2 hours in static air. The catalyst contained 0.06 w % Pd and 6 w % Re.

After drying, part of the catalyst was crushed to various particle sizes, about ˜0.5, ˜1 and ˜2 mm and the various fractions as well as uncrushed extrudates were evaluated for catalytic activity on converting furan to BDO and THF under operating conditions that varied with time but were identical for all catalysts.

### Comparative Example 3: Preparation of Pd/Re Catalyst for Catalytic Evaluation

For Comparative Example 3, an aqueous impregnation solution comprising a palladium-containing compound and a rhenium-containing compound was prepared by dissolving the target amount of tetraamine palladium nitrate (Pd(NH₃)₄(NO₃)₂ and perrhenic acid (HReO₄) into the target amount of demineralized water and homogenizing the solution for 30 seconds. The carbon support particles were loaded into a glass jar and the aqueous impregnation solution was then poured on the carbon support particles and homogenized using a rotary mixer for one hour. The palladium and rhenium impregnated carbon support particles were then transferred to a rotary bowl equipped with baffles and dried at 60° C. by means of an air dryer that heats the external wall of the bowl. The dried, palladium and rhenium impregnated carbon support particles were then transferred to a porcelain dish and dried in an oven set at 120° C. for 2 hours in static air. The catalyst contained 0.04 w % Pd and 4 w % Re.

After drying, part of the catalyst was crushed to various particle sizes, about ˜0.5, ˜1 and ˜2 mm and the various fractions as well as uncrushed particles were evaluated for catalytic activity on converting furan to BDO and THF under operating conditions that varied with time but were identical for all catalysts.

To evaluate catalytic performance of Example 5 and Comparative Example 3, a unit consisting of four microflow reactors operating in parallel was used. Each of the 9 mm tube reactors was filled with a mixture of 3 g catalyst of varying particle size and 3 g SiC (0.2 mm particle size). On top of this bed, a small plug of 0.8 mm SiC was placed to allow mixing and heating of the reactants prior to entering the catalytic bed. The reactors were placed in the unit. The catalyst was activated by heating it to 275° C. during 5 hours at atmospheric pressure under a H₂/N₂ stream 1 Nl/h/1 Nl/h. The temperature was kept at 275° C. for 2 hours and a H₂ flow of 1 Nl/h at 4 bar. The reactor was cooled to the reaction temperature (150° C.), maintaining the H₂ flow. Subsequently, the reactor was pressurized to 51 bar using a gas feed of 1.25 Nl/h H₂ (52 mmol/h) and 0.25 Nl/h N₂. Then the liquid feed was started (7 ml/h, 23% wt furan (21 mmol/h), 30.4% wt water (105 mmol/h) and 46.6% wt ethanol, density assumed from average of constituents). These flows resulted in a liquid WHSV of 2.1 h⁻¹ and a GHSV of 200 h⁻¹.

After the reactor, a gas liquid separation was performed at pressure and room temperature. These conditions were maintained for 72 hours, during which 6 liquid samples were obtained. Liquid samples were analysed on a GC (Agilent Technologies 6890N) equipped with an FID using diethylene glycoldiethylether as internal standard. This GC is equipped with a 50 meter CP-SIL5CB column with an inside diameter of 0.32 mm and a film thickness of 1.20 μm. No significant gas make was observed by the online GC.

For Example 5, the various catalyst particles were compared throughout the whole run and showed similar performance throughout 1000 h of run. For illustration, the performance observed at 150° C. and at WHSV=2/h after 260 h of run are reported below in Table 3 and confirm similar yields in BDO, THF and their molar ratio.

For Comparative Example 3, the various catalyst particles were compared throughout the whole run and showed very different performance for the uncrushed extrudates as compared to any crushed fraction. For illustration, the performance observed at 150′C and at WHSV=2/h after 70 h of run are reported below in Table 4 and confirm a severe increase in THF yield at the cost of BDO with extrudates. A similar, but much less pronounced effect is seen when the particles are crushed to a larger size only. This results in poor BDO selectivity for large particles, as expressed by the a high THF/BDO ratio.

TABLE 3
Yield and Selectivity of catalyst prepared according to Example 5.

| | | 0.2-0.6 mm | 0.85-1 mm | 2-2.4 mm | |
|---|---|---|---|---|---|
| particle | | (30-80 | (16-20 | (8-10 | 4 mm |
| size | | mesh) | mesh) | mesh) | (extrudate) |
| Yield | Mol % | 15.4 | 15.4 | 14.1 | 11.8 |
| in BDO | | | | | |
| Yield | Mol % | 13.5 | 13.3 | 11.5 | 13.5 |
| in THF | | | | | |
| ratio | Mol/mol | 0.88 | 0.86 | 0.82 | 1.14 |
| sum | Mol % | 28.9 | 28.7 | 25.6 | 25.4 |

TABLE 4
Yield and Selectivity of catalyst prepared
according to Comparative Example 3.

| | | 0.2-0.6 mm | >0.6 mm | 4 mm |
|---|---|---|---|---|
| particle size | | 30-80 mesh | >30 mesh | Extrudates |
| Yield in BDO | Mol % | 26.3 | 19.0 | 5.7 |
| Yield in THF | Mol % | 29.4 | 34.3 | 92.2 |
| ratio | Mol/mol | 1.12 | 1.81 | 16.24 |
| sum | Mol % | 55.7 | 53.4 | 97.8 |

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

1. A method for preparing a metal-impregnated, carbon-supported catalyst composition comprising:
providing a carbon support particle having a smallest dimension of greater than 0.5 millimeters;
contacting the carbon support particle with a basic aqueous impregnation solution comprising a base having a pK_{b} of at most 9 and at least one first metal-containing compound, wherein the first metal-containing compound comprises at least one first metal selected from groups 8, 9 and 10 of the periodic table, to form a first metal-impregnated carbon support particle; and
drying the first metal-impregnated carbon support particle.

2. The method of claim 1 further comprising:
subsequent to drying the first metal-impregnated carbon support particle, contacting the first metal-impregnated carbon support particle with an aqueous impregnation solution comprising at least one second metal-containing compound, wherein the second metal-containing compound comprises at least one second metal selected from groups 6 and 7 of the periodic table, to form a first and second metal-impregnated carbon support particle; and
drying the first and second metal-impregnated carbon support particle.

3. The method of claim 1 further comprising:
prior to contacting the carbon support particle with the basic aqueous impregnation solution comprising the base and the at least one first metal-containing compound, contacting the carbon support particle with an aqueous impregnation solution comprising at least one second metal-containing compound, wherein the second metal-containing compound comprises at least one second metal selected from groups 6 and 7 of the periodic table, to form a second metal-impregnated carbon support particle; and
drying the second metal-impregnated carbon support particle.

4. The method of claim 1, wherein the basic aqueous impregnation solution does not comprise ammonia and further comprises at least one second metal-containing compound, wherein the second metal-containing compound comprises at least one second metal selected from groups 6 and 7 of the periodic table.

5. The method of claim 1, wherein the first metal-containing compound comprises at least one first metal selected from ruthenium, rhodium, palladium, platinum, iridium and a combination thereof.

6. The method of claim 1, wherein the first metal-containing compound comprises palladium.

7. The method of claim 2, wherein the second metal-containing compound comprises at least one second metal selected from rhenium, molybdenum, tungsten and a combination thereof.

8. The method of claim 2 wherein the second metal-containing compound comprises rhenium.

9. The method of claim 1, wherein the base has a pK_{b} of at most 5.

10. The method of claim 1, wherein the base is ammonia.

11. The method of claim 1 further comprising:
prior to contacting the carbon support particle with the basic aqueous impregnation solution comprising the base and the at least one first metal-containing compound,
contacting the carbon support particle with a solution comprising a base having a pK_{b} of at most 9 to form a base-impregnated carbon support particle; and
drying the base-impregnated carbon support particle.

12. The process for the preparation of 1,4-butanediol and/or tetrahydrofuran comprising:
contacting furan, hydrogen and optionally water in the presence of a metal-impregnated, carbon-supported catalyst composition prepared in accordance with claim 1.
